(19) United States
(12) Patent Application Publication
(10) Pub. No.: US 2025/0019346 A1
(43) Pub. Date: Jan. 16, 2025

(54) Salts of Ruxolitinib and Crystalline Forms Thereof

(71) Applicant: Apotex Inc., Toronto (CA)

(72) Inventors: Fabio E. S. Souza, Brantford (CA); Alexander J. Stirk, Brantford (CA); Avedis Karadeolian, Brantford (CA); Allan W. Rey, Brantford (CA)

(21) Appl. No.: 18/711,252

(22) PCT Filed: Nov. 16, 2022
(86) PCT No.: PCT/CA2022/051690

§ 371 Date: May 17, 2024

(30) Foreign Application Priority Data
Nov. 19, 2021 (CA) 3139457

(51) Int. Cl. C07C 309/04; A61K 31/519; C07C 309/05; C07C 309/35; C07D 291/06; C07D 487/04

(52) U.S. Cl. CPC C07C 309/04; A61K 31/519; C07C 309/05; C07C 309/35; C07D 291/06; C07D 487/04; C07B 2200/13

(57) ABSTRACT

The present invention provides ruxolitinib salts and crystalline forms thereof, pharmaceutical compositions including these salts and crystalline forms thereof, and the use of these salts in the treatment of acute graft versus host disease, polycythemia vera, myelofibrosis, and atopic dermatitis.

### CROSS-REFERENCE TO RELATED APPLICATIONS

This application is the United States national phase of International Patent Application No. PCT/CA2022/051690 filed Nov. 16, 2022, and claims priority to Canadian Patent Application No. 3,139,457 filed Nov. 19, 2021, the disclosures of each of which are hereby incorporated by reference in their entireties.

### BACKGROUND

### Technical Field

The present invention is directed to novel salts of ruxolitinib, crystalline forms thereof, pharmaceutical compositions containing these salts, and their use in the treatment of diseases related to activity of Janus kinases.

### TECHNICAL CONSIDERATIONS

Ruxolitinib (1), or (R)-3-(4-(7H-pyrrolo [2,3d]pyrimidin-4-yl)-1H-pyrazol-1-yl)-3-cyclopentylpropanenitrile, in the form of its phosphate salt (1:1), is a Janus kinase inhibitor and the active pharmaceutical ingredient (API) in branded prescription pharmaceuticals JAKAFI® and OPZELURA®. JAKAFI® is used in the treatment of acute graft versus host disease, polycythemia vera, and myelofibrosis, and OPZELURA® is used in the treatment of eczema (atopic dermatitis).

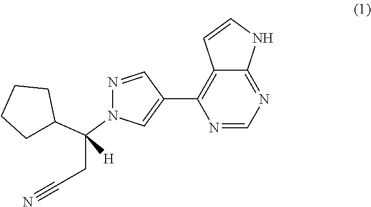 [SMILES: N#CC[C@H](C1CCCC1)n1cc(-c2ncnc3[nH]ccc23)cn1]

Ruxolitinib salts and crystalline forms thereof are reported in, for example, WO 2007/070514 A1, WO 2008/157208 A2, WO 2016/026974 A1, WO 2016/026975 A1, WO 2016/035014 A1, WO 2016/063294 A2, WO 2016/074650 A1, WO 2017/008772 A1, WO 2017/125097 A1, and WO 2019/241504 A1.

According to the review published by the U.S. Center for Drug Evaluation and Research (CDER) in connection with the approval of JAKAFI® (NDA 202192), the drug substance ruxolitinib phosphate has high solubility and high permeability, placing it in Class I of the Biopharmaceutics Classification System (BCS).

The solubility of individual salt and crystalline forms of a drug substance in an aqueous environment is an important aspect of their relative bioavailability, since the manner in which the salt or crystalline form dissolves can correspond to the amount of the drug substance that is available to be absorbed into the body to provide the intended therapeutic effect. One measure of solubility is intrinsic dissolution rate (IDR), which is defined as the dissolution rate of a substance under constant surface area conditions. For low solubility substances, higher IDR values can correlate with higher bioavailability following administration. However, if the goal is to establish bioequivalence to an approved form of a drug, such as ruxolitinib phosphate, substances with similar IDR values to the approved form are preferred. Alternatively, for the development of extended or sustained release products, forms exhibiting lower IDR values are often preferable since they can provide slower dissolution of the drug independent of the excipients used in the formulation.

Different salt and/or crystalline forms of the same compound may have different crystal packing, thermodynamic, spectroscopic, kinetic, surface, and mechanical properties. For example, different salts and/or crystalline forms may have different stability properties such that a particular form may be less sensitive to heat, relative humidity (RH) and/or light. As well, different salts and/or crystalline forms of a compound may be more susceptible to moisture uptake, resulting in a potential alteration of the chemical and/or physical stability. Different salts may exist in more than one crystalline form, which can cause complexity in ensuring the stability of a desired crystalline form in a drug product. Finally, different salts and/or crystalline forms of a compound may have different dissolution rates, thereby providing different pharmacokinetic parameters, which allow for specific forms to be used in order to achieve specific pharmacokinetic targets.

For example, a particular salt and/or crystalline form may provide more favourable performance in a topical formulation such as OPZELURA®, an oil-in-water, solubilized emulsion cream. According to WO 2011/146808 A2, factors considered in the production of a suitable topical formulation of ruxolitinib phosphate include appearance, spreadability, viscosity, and stability. Differences in properties between salts and/or crystalline forms of ruxolitinib can be exploited to provide alternative topical formulations having desired features. For example, differences in the solubility of a particular salt and/or crystalline form in an aqueous or oily environment can alter the stability or permeability of a particular topical formulation.

Therefore, there exists a need for novel salts and crystalline forms of ruxolitinib for use in providing improved drug products containing ruxolitinib, and commercially amenable processes for their manufacture.

### SUMMARY

The present invention provides salts comprising ruxolitinib and an acid having a sulfonyl group in the form of sulfamate ester (acesulfame) or an organic sulfonic acid selected from methanesulfonic acid, 1,2-ethanedisulfonic acid, and 1,5-naphthalenedisulfonic acid. The sulfonic acids used in the present invention are pharmaceutically acceptable acids. Acesulfame, in the form of acesulfamate potassium, is used as a sweetener in the food industry and as an inactive ingredient in drug products. Accordingly, it is expected that acesulfame can safely be used in materials intended for use in the preparation of pharmaceutical compositions intended for administration to humans.

The salts and crystalline forms of the present invention exhibit form stability at high temperature and high humidity and embodiments of the present invention exhibit similar or enhanced dissolution rates compared to ruxolitinib phosphate.

In addition, the processes for the manufacture of the ruxolitinib salts and crystalline forms of the present invention are efficient and industrially compatible.

Accordingly, in a first aspect of the present invention, there is provided a mesylate salt of ruxolitinib. In a preferred embodiment of the first aspect, the molar ratio of ruxolitinib to methanesulfonic acid is approximately 1:1. In a more preferred embodiment of the first aspect, the salt is characterized by a PXRD diffractogram comprising peaks, expressed in degrees 2θ (±0.2°), at 6.8°, 8.0°, and 11.1°. More preferably, the salt of the first aspect is characterized by a PXRD diffractogram further comprising at least three peaks, expressed in degrees 2θ (±0.2°), selected from the group consisting of: 11.7°, 13.1°, 16.2°, 17.8°, 19.3°, and 22.5°. In a further preferred embodiment of the first aspect, the PXRD diffractogram further comprises peaks, expressed in degrees 2θ (±0.2°), at 11.7°, 13.1°, 16.2°, 17.8°, 19.3°, and 22.5°. Preferably, the salt of the first aspect of the invention provides a PXRD diffractogram comprising peaks in substantially the same positions (±0.2° 2θ) as those shown in FIG. 1. In a further preferred embodiment of the first aspect, the salt is characterized by a DSC thermogram comprising an endothermic peak with a peak onset at approximately 163° C. and a peak maximum at approximately 166° C. Preferably, the salt of the first aspect is characterized by a DSC thermogram that is substantially the same in appearance as the DSC thermogram provided in FIG. 6.

In a second aspect of the present invention, there is provided an edisylate salt of ruxolitinib. In a preferred embodiment of the second aspect, the molar ratio of ruxolitinib to 1,2-ethanedisulfonic acid is approximately 1:1. In a more preferred embodiment of the second aspect, the salt is characterized by a PXRD diffractogram comprising peaks, expressed in degrees 2θ (±0.2°), at 4.1°, 8.3°, and 15.7°. More preferably, the salt of the second aspect is characterized by a PXRD diffractogram further comprising at least three peaks, expressed in degrees 2θ (±0.2°), selected from the group consisting of: 7.9°, 9.5°, 16.2°, 18.4°, 20.6, 17.7°, and 22.3°. In a further preferred embodiment of the second aspect, the PXRD diffractogram further comprises peaks, expressed in degrees 2θ (+) 0.2°, at 7.9°, 9.5°, 16.2°, 18.4°, 20.6, 17.7°, and 22.3°. Preferably, the salt of the second aspect of the invention provides a PXRD diffractogram comprising peaks in substantially the same positions (±0.2° 2θ) as those shown in FIG. 2. In a further preferred embodiment of the second aspect, the salt is characterized by a DSC thermogram comprising an endothermic peak with a peak onset at approximately 148° C. and a peak maximum at approximately 157° C. Preferably, the salt of the second aspect is characterized by a DSC thermogram that is substantially the same in appearance as the DSC thermogram provided in FIG. 7.

In a third aspect of the present invention, there is provided a napadisylate salt of ruxolitinib. In a preferred embodiment of the third aspect, the molar ratio of ruxolitinib to 1,5-naphthalenedisulfonic acid is approximately 1:1. In a more preferred embodiment of the third aspect, the salt is characterized by a PXRD diffractogram comprising peaks, expressed in degrees 2θ (±0.2°), at 7.2°, 9.5°, and 15.7°. More preferably, the salt of the third aspect is characterized by a PXRD diffractogram further comprising at least three peaks, expressed in degrees 2θ (±0.2°), selected from the group consisting of: 12.0°, 14.3°, 16.7°, 17.3°, 18.8°, and 19.4°. In a further preferred embodiment of the third aspect, the PXRD diffractogram further comprises peaks, expressed in degrees 2θ (±0.2°), at 12.0°, 14.3°, 16.7°, 17.3°, 18.8°, and 19.4°. Preferably, the salt of the third aspect of the invention provides a PXRD diffractogram comprising peaks in substantially the same positions (±0.2° 2θ) as those shown in FIG. 3. In a further preferred embodiment of the third aspect, the salt is characterized by a DSC thermogram comprising an endothermic peak with a peak onset at approximately 154° C. and a peak maximum at approximately 165° C. Preferably, the salt of the third aspect is characterized by a DSC thermogram that is substantially the same in appearance as the DSC thermogram provided in FIG. 8.

In a fourth aspect of the present invention, there is provided an acesulfamate salt of ruxolitinib. In a preferred embodiment of the fourth aspect, the molar ratio of ruxolitinib to acesulfame is approximately 1:1. In a first preferred embodiment of the fourth aspect, the salt is characterized by a PXRD diffractogram comprising peaks, expressed in degrees 2θ (±0.2°), at 4.1°, 9.0°, and 22.5°. More preferably, the salt of this embodiment is characterized by a PXRD diffractogram further comprising at least three peaks, expressed in degrees 2θ (±0.2°), selected from the group consisting of: 8.4°, 12.8°, 13.5°, 15.8°, 20.3°, and 25.5°. More preferably, the PXRD diffractogram of this embodiment further comprises peaks, expressed in degrees 2θ (±0.2°), at 8.4°, 12.8°, 13.5°, 15.8°, 20.3°, and 25.5°. Preferably, the salt of this embodiment provides a PXRD diffractogram comprising peaks in substantially the same positions (±0.2° 2θ) as those shown in FIG. 4. The salt of this embodiment is preferably characterized by a DSC thermogram comprising an endothermic peak with a peak onset at approximately 112° C. and a peak maximum at approximately 121° C. Preferably, the salt of this embodiment is characterized by a DSC thermogram that is substantially the same in appearance as the DSC thermogram provided in FIG. 9. In a second preferred embodiment of the fourth aspect, the salt is characterized by a PXRD diffractogram comprising peaks, expressed in degrees 2θ (±0.2°), at 10.8°, 11.4°, and 13.0°. More preferably, the salt of this embodiment is characterized by a PXRD diffractogram further comprising at least three peaks, expressed in degrees 2θ (±0.2°), selected from the group consisting of: 4.4°, 8.7°, 14.0°, 15.9°, 20.5°, and 21.6°. More preferably, the PXRD diffractogram of this embodiment further comprises peaks, expressed in degrees 2θ (±0.2°), at 4.4°, 8.7°, 14.0°, 15.9°, 20.5°, and 21.6°. Preferably, the salt of this embodiment provides a PXRD diffractogram comprising peaks in substantially the same positions (±0.2° 2θ) as those shown in FIG. 5. The salt of this embodiment is preferably characterized by a DSC thermogram comprising an endothermic peak with a peak onset at approximately 77° C. and a peak maximum at approximately 81° C. Preferably, the salt of this embodiment is characterized by a DSC thermogram that is substantially the same in appearance as the DSC thermogram provided in FIG. 10.

In a fifth aspect of the present invention, there is provided a pharmaceutical composition comprising a salt of ruxolitinib according to the first, second, third, or fourth aspects of the invention, and one or more pharmaceutically acceptable excipients. Preferably, the pharmaceutical composition is in the form of a tablet or an oil-in-water cream emulsion. Preferably, the pharmaceutical composition of the fifth aspect is a tablet that comprises an amount of the ruxolitinib salt of the first, second, third, or fourth aspects that is equivalent to 5 mg, 10 mg, 15 mg, 20 mg, or 25 mg of ruxolitinib free base. Preferably, the pharmaceutical composition of the fifth aspect is an oil-in-water cream that comprises an amount of the ruxolitinib salt of the first, second, third, or fourth aspects that is equivalent to 15 mg of ruxolitinib free base/gram of cream.

In a sixth aspect of the present invention, there is provided the use of a salt of ruxolitinib according to the first, second, third, or fourth aspects of the invention, or the pharmaceutical tablet composition of the fifth aspect of the invention, in the treatment of a disorder selected from the group consisting of acute graft versus host disease, polycythemia vera, and myelofibrosis. In a further embodiment of the sixth aspect, there is provided the use of a salt of ruxolitinib according to the first, second, third, or fourth aspects of the invention, or the pharmaceutical cream composition of the fifth aspect of the invention, in the treatment of atopic dermatitis.

Other aspects and features of the present invention will become apparent to those ordinarily skilled in the art upon review of the following description of specific embodiments of the invention in conjunction with the accompanying figures.

### BRIEF DESCRIPTION OF THE DRAWINGS

The terms Fig., Figs., Figure, and Figures are used interchangeably in the specification to refer to the corresponding figures in the drawings.

Embodiments of the present invention are described, by way of example only, with reference to the attached Figures.

FIG. 1 is a representative PXRD diffractogram of ruxolitinib mesylate Form APO-I as prepared in Example 1.

FIG. 2 is a representative PXRD diffractogram of ruxolitinib edisylate Form APO-I as prepared in Example 2.

FIG. 3 is a representative PXRD diffractogram of ruxolitinib napadisylate Form APO-I as prepared in Example 3.

FIG. 4 is a representative PXRD diffractogram of ruxolitinib acesulfamate Form APO-I as prepared in Example 4.

FIG. 5 is a representative PXRD diffractogram of ruxolitinib acesulfamate Form APO-II as prepared in Example 5.

FIG. 6 is a representative DSC thermogram of ruxolitinib mesylate Form APO-I as prepared in Example 1.

FIG. 7 is a representative DSC thermogram of ruxolitinib edisylate Form APO-I as prepared in Example 2.

FIG. 8 is a representative DSC thermogram of ruxolitinib napadisylate Form APO-I as prepared in Example 3.

FIG. 9 is a representative DSC thermogram of ruxolitinib acesulfamate Form APO-I as prepared in Example 4.

FIG. 10 is a representative DSC thermogram of ruxolitinib acesulfamate Form APO-II as prepared in Example 5.

### DETAILED DESCRIPTION

The present invention provides ruxolitinib salts and crystalline forms thereof comprising an acid having a sulfonyl moiety in the form of sulfamate ester (acesulfame) or an organic sulfonic acid selected from methanesulfonic acid, 1,2-ethanedisulfonic acid and 1,5-naphthalenedisulfonic acid. The organic sulfonic acids used in the present invention are considered class two acids according to a notable reference book on the pharmaceutical acceptability of salts: P. Heinrich Stahl, Camille G. Wermuth (Eds.), Handbook of Pharmaceutical Salts Properties, Selection, and Use; 2002. Class two acids are classified by Stahl et al. as those that are not naturally occurring, but, so far, during their profuse application have shown low toxicity and good tolerability. Acesulfame, in the form of its potassium salt, is used as a sweetener in the food industry and is also included in both the U.S. Food & Drug Administration's (FDA's) Substances Added to Food inventory (formerly Everything Added to Food in the United States (EAFUS)) list and the Inactive Ingredient Database (IID). The Substances Added to Food inventory contains approximately 4,000 substances, and includes information on food additives, colour additives, Generally Recognized As Safe (GRAS) substances, and prior-sanctioned substances. The IID list provides information on inactive ingredients present in FDA-approved drug products. Once an inactive ingredient has appeared in an approved drug product, the inactive ingredient is not considered new, and may require a less extensive review the next time it is included in a new drug product. In addition to pharmaceutical acceptability, the acesulfamate counterion may offer a dual purpose by imparting a sweet taste that could be exploited in certain pharmaceutical formulations such as oral solutions.

The present invention provides ruxolitinib salts and crystalline forms thereof providing improved properties over known salts of ruxolitinib. Properties that differ between the invention and known forms of ruxolitinib include the following: packing properties such as molar volume, density and hygroscopicity; thermodynamic properties such as melting point and solubility; kinetic properties such as dissolution rate and chemical/polymorphic stability; surface properties such as crystal habit; and/or mechanical properties such as hardness, tensile strength, cohesiveness, compactibility, tableting, handling, flow, and blending.

Additionally, processes for the manufacture of ruxolitinib and crystalline forms thereof of the present invention are efficient and industrially compatible, using Class 3 solvents established by the ICH (International Council for Harmonization of Technical Requirements for Pharmaceuticals for Human Use) as having low toxicity.

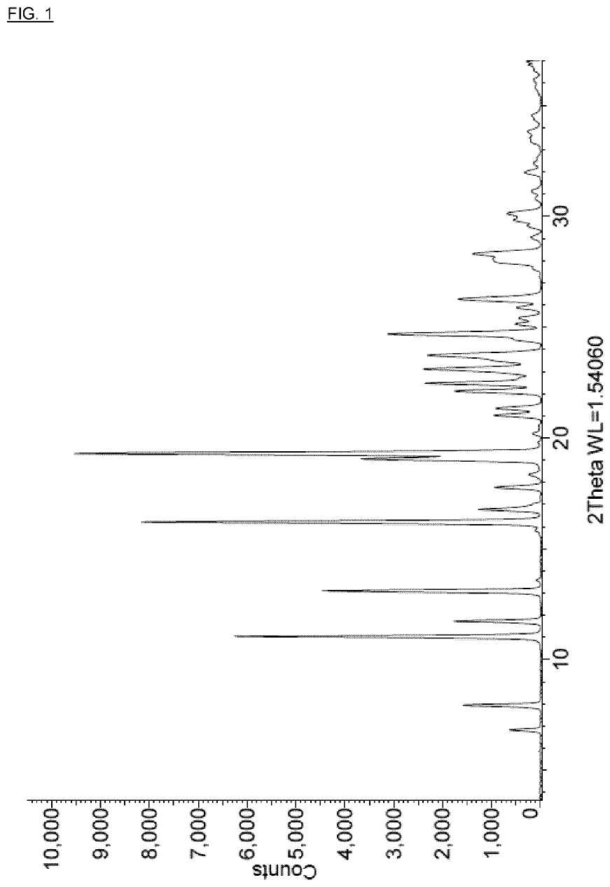

Depending on the manner in which the crystalline forms of the present invention are prepared, and the methodology and instrument used for PXRD analysis, the intensity of a given peak observed in a PXRD diffractogram of a crystalline form may vary when compared to the same peak in the representative PXRD diffractograms provided in FIGS. 1 to 5. Thus, differences in relative peak intensities between peaks in a PXRD diffractogram for a given crystalline form may be observed when compared to the relative peak intensities of the peaks in the representative PXRD diffractograms of FIGS. 1 to 5. Any such differences may be due, in part, to the preferred orientation of the sample and its deviation from the ideal random sample orientation, the preparation of the sample for analysis, and the methodology applied for the analysis. Such variations are known and understood by a person of skill in the art, and any such variations do not depart from the invention disclosed herein.

In addition to the differences in relative peak intensities that may be observed in comparison to the representative PXRD diffractograms provided in FIGS. 1 to 5, it is understood that individual peak positions may vary between ±0.2° 2θ from the values observed in the representative PXRD diffractograms provided in FIGS. 1 to 5 for the crystalline forms of the invention, or listed in Tables 1 to 5. Such variations are known and understood by a person of skill in the art, and any such variations do not depart from the invention disclosed herein.

Further, depending on the instrument used for X-ray analysis and its calibration, uniform offsets in the peak position of each peak in a PXRD diffractogram of greater that 0.2° 2θ may be observed when compared to the representative PXRD diffractograms provided in FIGS. 1 to 5. Thus, PXRD diffractograms of the crystalline forms of the present invention may, in some circumstances, display the same relative peak positions as observed in the representative PXRD diffractograms provided in FIGS. 1 to 5, with the exception that each peak is offset in the same direction, and by approximately the same amount, such that the overall PXRD diffractogram is substantially the same in appearance as the PXRD diffractograms of FIGS. 1 to 5, with the exception of the uniform offset in peak positions. The observation of any such uniform peak shift in a PXRD diffractogram does not depart from the invention disclosed herein given that the relative peak positions of the individual peaks within the PXRD diffractogram remain consistent with the relative peak positions observed in the PXRD diffractograms of FIGS. 1 to 5.

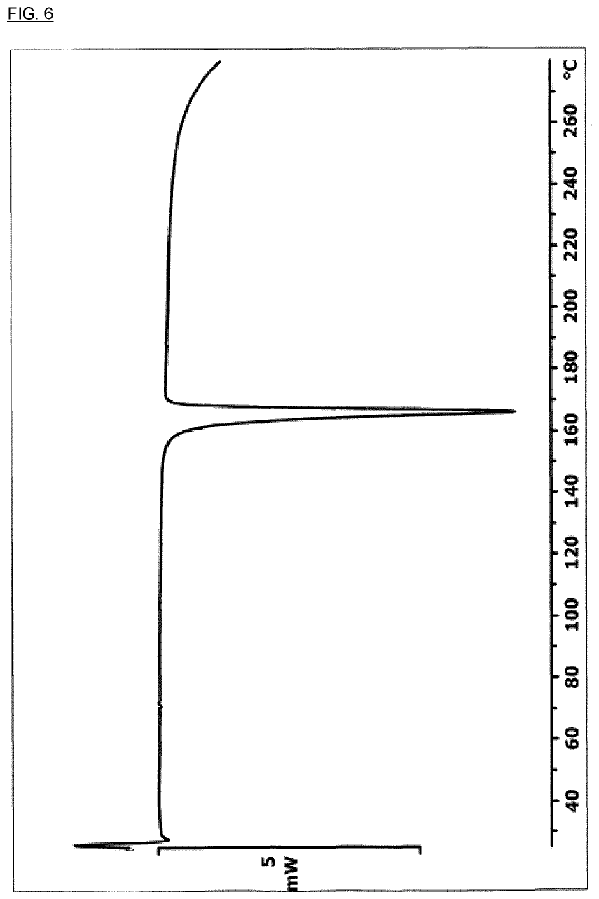

Depending on the manner in which the crystalline forms are prepared, the methodology and instrument used for DSC analysis, it is understood that peaks corresponding with thermal events in a DSC thermogram may vary between +2° C. from the values observed in the representative DSC thermograms provided in FIGS. 6 to 10 and described herein. Such variations are known and understood by a person of skill in the art, and any such variations do not depart from the invention disclosed herein.

As used herein, the term ‘crystalline form’ refers to a ruxolitinib salt of fixed composition with a particular arrangement of components in its crystal lattice, and which may be identified by physical characterization methods such as PXRD. As used herein, the term crystalline form is intended to include single-component and multiple-component crystalline forms of a ruxoltinib salt. Single-component forms of a ruxolitinib salt, consist solely of ruxolitinib and the counterion in the repeating unit of the crystal lattice. Multiple-component forms of a ruxolitinib salt include solvates (and hydrates) of a ruxolitinib salt wherein a solvent (or water) is also incorporated into the crystal lattice.

As used herein, the term ‘volumes’ refers to the parts of solvent or liquids by volume (mL) with respect to the weight of solute (g). For example, when an experiment is conducted using 1 g of ruxolitinib and 10 mL of solvent, it is said that 10 volumes of solvent are used.

As used herein, when referring to water content, the term “weight percentage” (wt %) refers to the ratio: weight water/weight solution in case of solution or weight water/weight sample in case of solid, expressed as a percentage.

As used herein, the term “room temperature” refers to a temperature in the range of 20° C. to 25° C.

Unless defined otherwise herein, the term “approximately”, when used in reference to molar ratios, allows for a variance of plus or minus 10%.

When describing the embodiments of the present invention there may be a common variance to a given temperature or time that would be understood or expected by the person skilled in the art to provide substantially the same result. For example, when reference is made to a particular temperature, it is to be understood by the person skilled in the art that there is an allowable variance of +5° C. associated with that temperature. When reference is made to a particular time, it is to be understood that there is an allowable variance of ±10 minutes when the time is one or two hours, and +1 hour when longer periods of time are referenced.

In one embodiment of the present invention, there is provided a new salt of ruxolitinib, ruxolitinib mesylate Form APO-I, wherein the molar ratio of ruxolitinib to methanesulfonic acid is approximately 1:1.

Ruxolitinib mesylate Form APO-I can be characterized by a PXRD diffractogram comprising, among other peaks, characteristic peaks, expressed in degrees 2θ (±0.2°), at 6.8°, 8.0°, and 11.1°. Preferably, the PXRD diffractogram further comprises at least three peaks, expressed in degrees 2θ (±0.2°), selected from the group consisting of 11.7°, 13.1°, 16.2°, 17.8°, 19.3°, and 22.5°. More preferably, the PXRD diffractogram further comprises peaks, expressed in degrees 2θ (±0.2°), at 11.7°, 13.1°, 16.2°, 17.8°, 19.3°, and 22.5°. PXRD studies of uncapped samples of ruxolitinib mesylate Form APO-I maintained in a 40° C./75% RH (relative humidity) stability chamber for at least 8 months showed that no change in the crystalline form occurred.

An illustrative PXRD diffractogram of ruxolitinib mesylate Form APO-I, as prepared in Example 1, is shown in FIG. 1. A peak listing, comprising representative peaks from the PXRD diffractogram in FIG. 1, and their relative intensities, is provided in Table 1. Although illustrative of the PXRD diffractogram that is provided for the ruxolitinib mesylate Form APO-I of the present invention, the relative intensities of the peaks are variable. Thus, depending on a particular sample, the prominence or relative intensity of the peaks observed may differ from those in the illustrative PXRD diffractogram and peak listing.

TABLE 1
Relative peak intensities of ruxolitinib
mesylate Form APO-I from FIG. 1

| Angle (2θ) | Relative intensity (%) |
|---|---|
| 6.83 | 6.2 |
| 7.95 | 15.9 |
| 11.05 | 64.2 |
| 11.74 | 18.3 |
| 13.10 | 46.3 |
| 16.21 | 84.1 |
| 16.77 | 13.3 |
| 17.77 | 9.7 |
| 19.31 | 100.0 |
| 21.02 | 9.9 |
| 22.11 | 18.3 |
| 22.47 | 24.7 |
| 23.11 | 24.9 |
| 23.73 | 23.8 |
| 24.69 | 32.3 |

An illustrative DSC thermogram of ruxolitinib mesylate Form APO-I is shown in FIG. 6. The DSC thermogram may be further characterized by an endothermic peak with a peak onset at approximately 163° C. and a peak maximum at approximately 166° C.

As described in Example 1, ruxolitinib mesylate Form APO-I can be prepared by combining ruxolitinib free base with approximately 1.5 mole equivalents of methanesulfonic acid in a suitable solvent, preferably acetone, and maintaining the mixture at a suitable temperature, preferably room temperature, for a suitable time, preferably between 12 and 20 hours. The resulting suspension is isolated and dried, if necessary, preferably in vacuo and at room temperature and maintaining the mixture at a suitable temperature, preferably room temperature, for a suitable time, preferably between 12 and 20 hours. The resulting suspension is isolated and dried, if necessary, preferably in vacuo and at room temperature.

Preferably, ruxolitinib mesylate Form APO-I is prepared by the method of Example 1.1, by combining ruxolitinib free base with approximately 1 mole equivalents of methanesulfonic acid, for example from 0.97 to 1.06 mole equivalents, in a suitable solvent, preferably a mixture of acetone and ethyl acetate containing water. A suitable solvent mixture includes a range of relative proportions of ethyl acetate and acetone, with a preferred total volume of from 8 to 18.5 volumes with respect to ruxolitinib free base. Preferably, the system contains an amount of water sufficient to afford a free-flowing suspension during crystallization, typically ranging from about 5 wt % to about 10 wt %. Preferably, amounts of acetone, ethyl acetate and water sufficient to maintain a monophasic system are used. Following treatment with methanesulfonic acid, which is preferably added in divided portions, the mixture is maintained at a suitable temperature, preferably room temperature, for a suitable time, preferably between 12 and 20 hours. The resulting suspension is isolated and dried, if necessary, preferably in vacuo and at room temperature.

In a second embodiment of the present invention, there is provided a new salt of ruxolitinib, ruxolitinib edisylate Form APO-I, wherein the molar ratio of ruxolitinib to 1,2-ethanedisulfonic acid is approximately 1:1.

Ruxolitinib edisylate Form APO-I can be characterized by a PXRD diffractogram comprising, among other peaks, characteristic peaks, expressed in degrees 2θ (±0.2°), at 4.1°, 8.3°, and 15.7°. Preferably, the PXRD diffractogram further comprises at least three peaks, expressed in degrees 2θ (±0.2°), selected from the group consisting of 7.9°, 9.5°, 16.2°, 18.4°, 20.6, 17.7°, and 22.3°. More preferably, the PXRD diffractogram further comprises peaks, expressed in degrees 2θ (±0.2°), at 7.9°, 9.5°, 16.2°, 18.4°, 20.6, 17.7°, and 22.3°. PXRD studies of uncapped samples of ruxolitinib edisylate Form APO-I maintained in a 40° C./75% RH stability chamber for at least 5 months showed that no change in the crystalline form occurred.

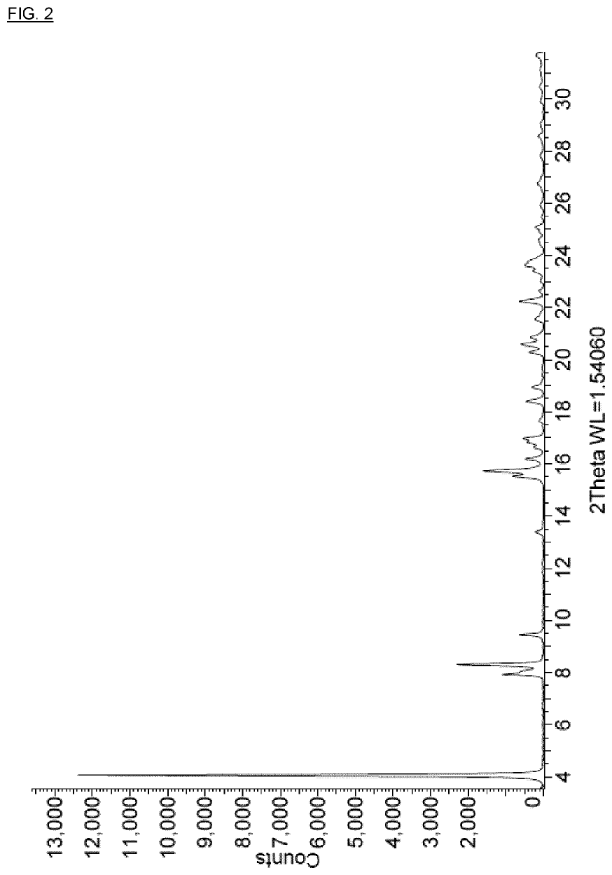

An illustrative PXRD diffractogram of ruxolitinib edisylate Form APO-I, as prepared in Example 2, is shown in FIG. 2. A peak listing, comprising representative peaks from the PXRD diffractogram in FIG. 2, and their relative intensities, is provided in Table 2. Although illustrative of the PXRD diffractogram that is provided for the ruxolitinib edisylate Form APO-I of the present invention, the relative intensities of the peaks are variable. Thus, depending on a particular sample, the prominence or relative intensity of the peaks observed may differ from those in the illustrative PXRD diffractogram and peak listing.

TABLE 2
Relative peak intensities of ruxolitinib
edisylate Form APO-I from FIG. 2

| Angle (2θ) | Relative intensity (%) |
|---|---|
| 4.07 | 100.0 |
| 7.93 | 8.9 |
| 8.31 | 18.6 |
| 9.45 | 5.4 |
| 15.53 | 6.7 |
| 15.74 | 13.5 |
| 16.20 | 3.9 |
| 18.40 | 3.9 |
| 18.94 | 2.7 |
| 20.59 | 4.8 |
| 22.25 | 5.7 |

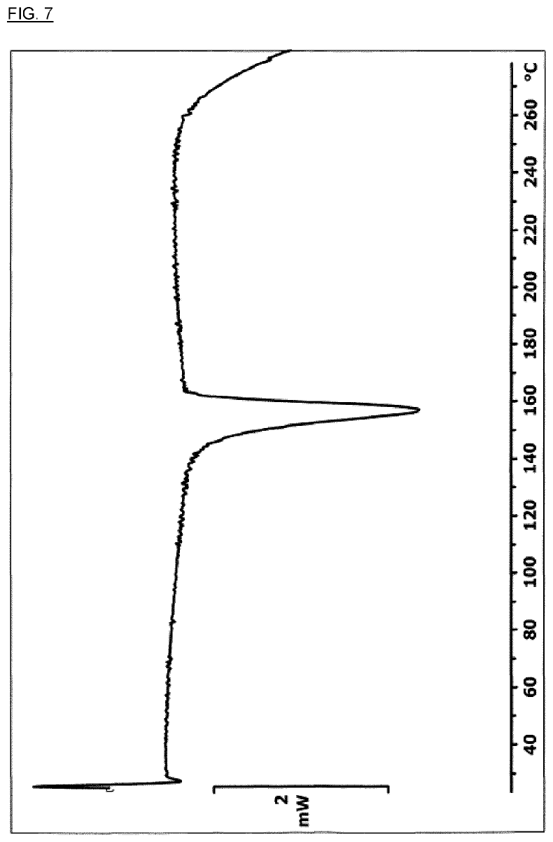

An illustrative DSC thermogram of ruxolitinib edisylate Form APO-I is shown in FIG. 7. The DSC thermogram may be further characterized by an endothermic peak with a peak onset at approximately 148° C. and a peak maximum at approximately 157° C.

As described in Example 2, ruxolitinib edisylate Form APO-I can be prepared by combining ruxolitinib free base with approximately 0.5 mole equivalents of 1,2-ethanedisulfonic acid dihydrate in a mixture of water and a suitable water-miscible solvent, preferably acetone, and maintaining the mixture at a suitable temperature, preferably room temperature, for a suitable time, preferably between 12 and 20 hours. The resulting suspension is isolated and dried, if necessary, preferably in vacuo and at room temperature.

In a third embodiment of the present invention, there is provided a new salt of ruxolitinib, ruxolitinib napadisylate Form APO-I, wherein the molar ratio of ruxolitinib to 1,5-naphthalenedisulfonic acid is approximately 1:1.

Ruxolitinib napadisylate Form APO-I can be characterized by a PXRD diffractogram comprising, among other peaks, characteristic peaks, expressed in degrees 2θ (±0.2°), at 7.2°, 9.5°, and 15.7°. Preferably, the PXRD diffractogram further comprises at least three peaks, expressed in degrees 2θ (±0.2°), selected from the group consisting of 12.0°, 14.3°, 16.7°, 17.3°, 18.8°, and 19.4°. More preferably, the PXRD diffractogram further comprises peaks, expressed in degrees 2θ (±0.2°), at 12.0°, 14.3°, 16.7°, 17.3°, 18.8°, and 19.4°. PXRD studies of uncapped samples of ruxolitinib napadisylate Form APO-I maintained in a 40° C./75% RH stability chamber for at least 5 months showed that no change in the crystalline form occurred.

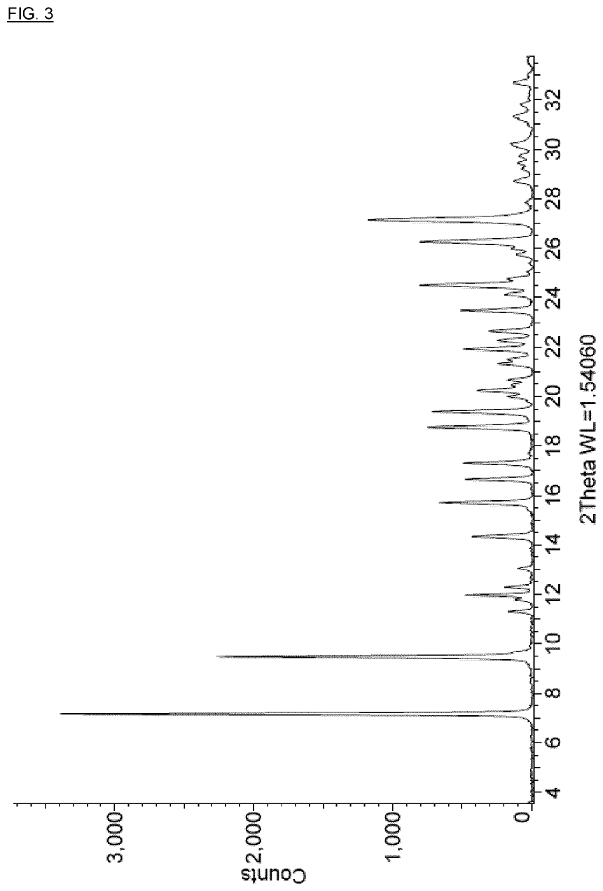

An illustrative PXRD diffractogram of ruxolitinib napadisylate Form APO-I, as prepared in Example 3, is shown in FIG. 3. A peak listing, comprising representative peaks from the PXRD diffractogram in FIG. 3, and their relative intensities, is provided in Table 3. Although illustrative of the PXRD diffractogram that is provided for the ruxolitinib napadisylate Form APO-I of the present invention, the relative intensities of the peaks are variable. Thus, depending on a particular sample, the prominence or relative intensity of the peaks observed may differ from those in the illustrative PXRD diffractogram and peak listing.

TABLE 3
Relative peak intensities of ruxolitinib
napadisylate Form APO-I from FIG. 3

| Angle (2θ) | Relative intensity (%) |
|---|---|
| 7.18 | 100.0 |
| 9.49 | 68.2 |
| 11.97 | 14.8 |
| 14.34 | 13.2 |
| 15.72 | 20.3 |
| 16.66 | 14.6 |
| 17.31 | 15.7 |
| 18.76 | 23.5 |
| 19.39 | 22.7 |
| 21.92 | 16.1 |
| 23.48 | 16.8 |
| 24.50 | 25.9 |
| 26.26 | 26.2 |
| 27.14 | 38.5 |

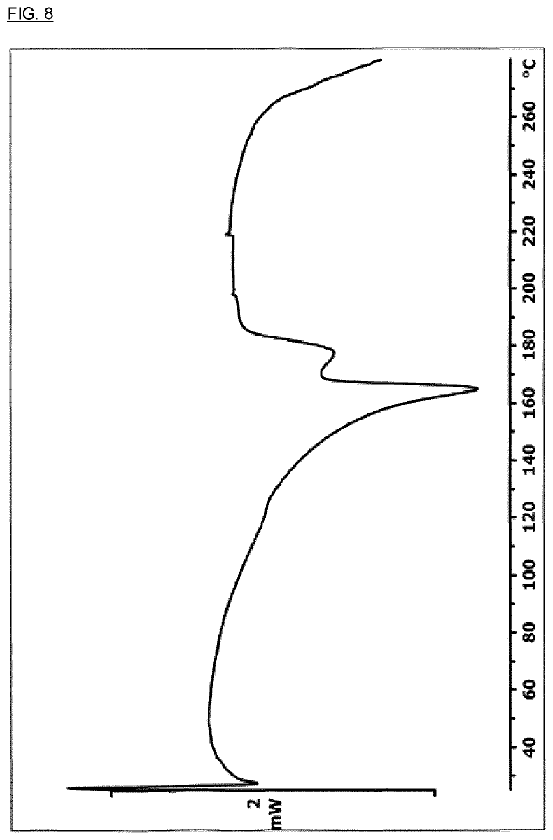

An illustrative DSC thermogram of ruxolitinib napadisylate Form APO-I is shown in FIG. 8. The DSC thermogram may be further characterized by an endothermic peak with a peak onset at approximately 154° C. and a peak maximum at approximately 165° C.

As described in Example 3, ruxolitinib napadisylate Form APO-I can be prepared by combining ruxolitinib free base with approximately 0.5 mole equivalents of 1,5-naphthalenedisulfonic acid tetrahydrate in a mixture of water and a suitable water-miscible solvent, preferably acetone, and maintaining the mixture at an elevated temperature, preferably between approximately 30° C. and 50° C., for a suitable time, preferably between approximately 0.5 and 2 hours, followed by cooling, if necessary. The resulting suspension is isolated and dried, if necessary, preferably in vacuo and at room temperature.

In a fourth embodiment of the present invention, there is provided a new salt of ruxolitinib, ruxolitinib acesulfamate Form APO-I, wherein the molar ratio of ruxolitinib to acesulfame is approximately 1:1.

Ruxolitinib acesulfamate Form APO-I can be characterized by a PXRD diffractogram comprising, among other peaks, characteristic peaks, expressed in degrees 2θ (±0.2°), at 4.1°, 9.0°, and 22.5°. Preferably, the PXRD diffractogram further comprises at least three peaks, expressed in degrees 2θ (±0.2°), selected from the group consisting of 8.4°, 12.8°, 13.5°, 15.8°, 20.3°, and 25.5°. More preferably, the PXRD diffractogram further comprises peaks, expressed in degrees 2θ (±0.2°), at 8.4°, 12.8°, 13.5°, 15.8°, 20.3°, and 25.5°. PXRD studies of uncapped samples of ruxolitinib acesulfamate Form APO-I maintained in a 40° C./75% RH stability chamber for 8 weeks showed that no change in the crystalline form occurred.

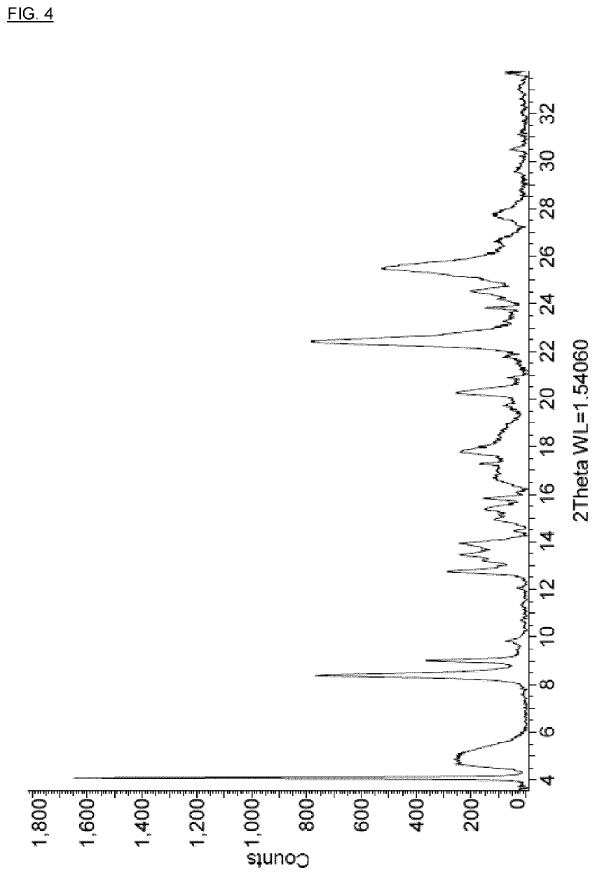

An illustrative PXRD diffractogram of ruxolitinib acesulfamate Form APO-I, as prepared in Example 4, is shown in FIG. 4. A peak listing, comprising representative peaks from the PXRD diffractogram in FIG. 4, and their relative intensities, is provided in Table 4. Although illustrative of the PXRD diffractogram that is provided for the ruxolitinib acesulfamate Form APO-I of the present invention, the relative intensities of the peaks are variable. Thus, depending on a particular sample, the prominence or relative intensity of the peaks observed may differ from those in the illustrative PXRD diffractogram and peak listing.

TABLE 4
Relative peak intensities of ruxolitinib
acesulfamate Form APO-I from FIG. 4

| Angle (2θ) | Relative intensity (%) |
|---|---|
| 4.08 | 100.0 |
| 8.38 | 46.7 |
| 9.02 | 22.4 |
| 12.76 | 17.1 |
| 13.46 | 14.8 |
| 13.93 | 15.4 |
| 15.82 | 10.4 |
| 17.80 | 13.8 |
| 20.29 | 14.9 |
| 22.45 | 47.6 |
| 25.50 | 31.7 |

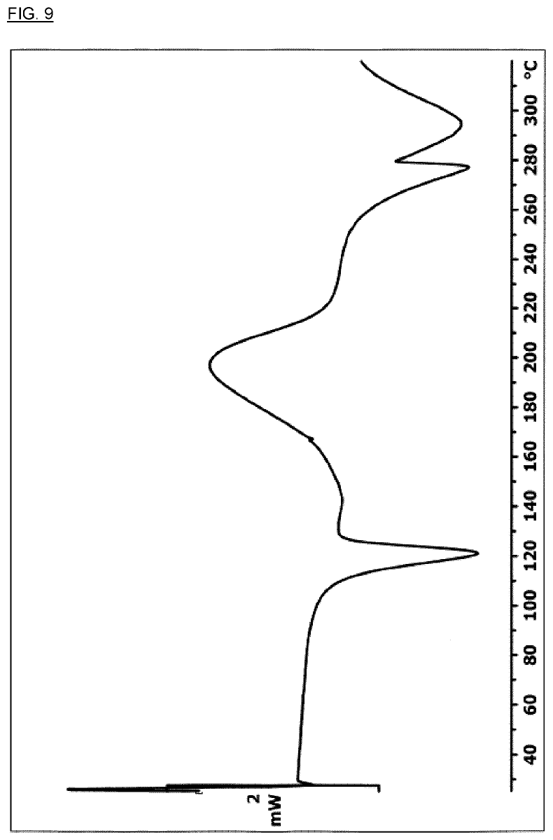

An illustrative DSC thermogram of ruxolitinib acesulfamate Form APO-I is shown in FIG. 9. The DSC thermogram may be further characterized by an endothermic peak with a peak onset at approximately 112° C. and a peak maximum at approximately 121° C.

As described in Example 4, ruxolitinib acesulfamate Form APO-I can be prepared by combining approximately equimolar amounts of ruxolitinib free base and acesulfame in a suitable solvent, preferably ethyl acetate, and maintaining the mixture at an elevated temperature, preferably between approximately 55° C. and 75° C., for a suitable time, preferably between approximately 0.5 and 2 hours followed by cooling, if necessary. The resulting suspension is isolated and dried, if necessary, preferably in vacuo and at room temperature.

In a fifth embodiment of the present invention, there is provided a new salt of ruxolitinib, ruxolitinib acesulfamate Form APO-II, wherein the molar ratio of ruxolitinib to acesulfame is approximately 1:1.

Ruxolitinib acesulfamate Form APO-II can be characterized by a PXRD diffractogram comprising, among other peaks, characteristic peaks, expressed in degrees 2θ (±0.2°), at 10.8°, 11.4°, and 13.0°. Preferably, the PXRD diffractogram further comprises at least three peaks, expressed in degrees 2θ (±0.2°), selected from the group consisting of 4.4°, 8.7°, 14.0°, 15.9°, 20.5°, and 21.6°. More preferably, the PXRD diffractogram further comprises peaks, expressed in degrees 2θ (±0.2°), at 4.4°, 8.7°, 14.0°, 15.9°, 20.5°, and 21.6°. PXRD studies of uncapped samples of ruxolitinib acesulfamate Form APO-II maintained in a 40° C./75% RH stability chamber for at least 5 months showed that no change in the crystalline form occurred.

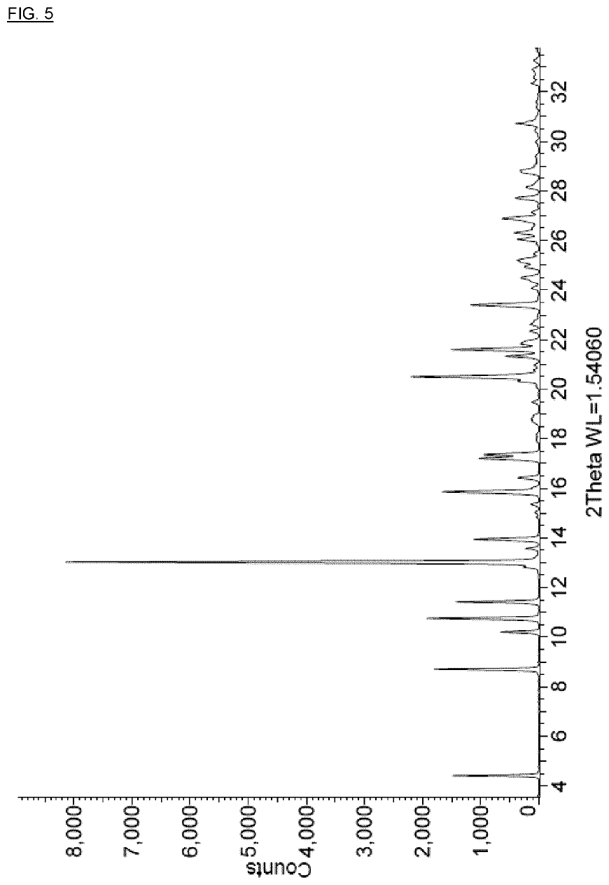

An illustrative PXRD diffractogram of ruxolitinib acesulfamate Form APO-II, as prepared in Example 5, is shown in FIG. 5. A peak listing, comprising representative peaks from the PXRD diffractogram in FIG. 5, and their relative intensities, is provided in Table 5. Although illustrative of the PXRD diffractogram that is provided for the ruxolitinib acesulfamate Form APO-II of the present invention, the relative intensities of the peaks are variable. Thus, depending on a particular sample, the prominence or relative intensity of the peaks observed may differ from those in the illustrative PXRD diffractogram and peak listing.

TABLE 5
Relative peak intensities of ruxolitinib
acesulfamate Form APO-II from FIG. 5

| Angle (2θ) | Relative intensity (%) |
|---|---|
| 4.42 | 17.0 |
| 8.71 | 21.2 |
| 10.21 | 7.9 |
| 10.76 | 23.3 |
| 11.43 | 17.0 |
| 13.03 | 100.0 |
| 13.95 | 13.5 |
| 15.86 | 20.1 |
| 17.21 | 12.7 |
| 20.50 | 29.4 |
| 21.60 | 19.5 |
| 24.00 | 14.9 |

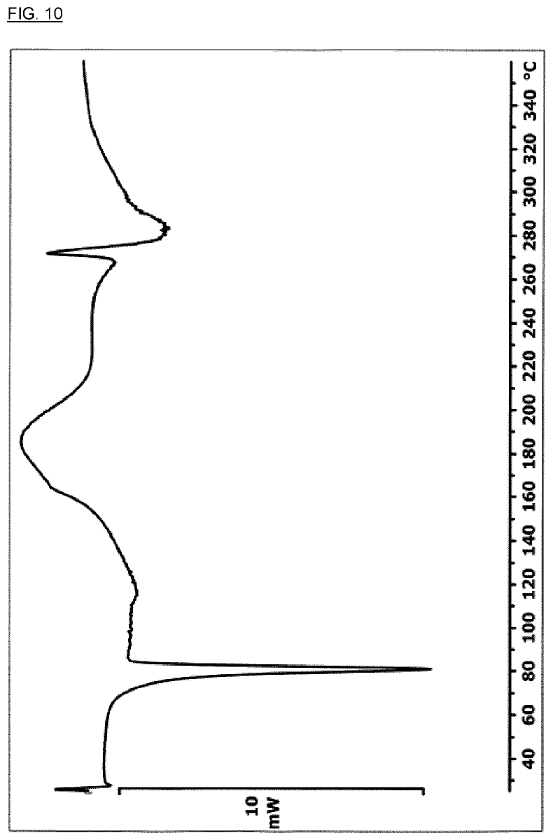

An illustrative DSC thermogram of ruxolitinib acesulfamate Form APO-II is shown in FIG. 10. The DSC thermogram may be further characterized by an endothermic peak with a peak onset at approximately 77° C. and a peak maximum at approximately 81° C.

As described in Example 5, ruxolitinib acesulfamate Form APO-II can be prepared by combining approximately equimolar amounts of ruxolitinib free base and acesulfame in a mixture of water and a suitable water-miscible solvent, preferably isopropanol, and maintaining the mixture a suitable temperature, preferably room temperature, for a suitable time, preferably between approximately 4 and 6 hours. The resulting suspension is isolated and dried, if necessary, preferably in vacuo and at room temperature.

In a sixth embodiment of the invention, there is provided a pharmaceutical composition comprising one or more ruxolitinib salt(s) selected from the group consisting of ruxolitinib mesylate, ruxolitinib edisylate, ruxolitinib napadisylate, or ruxolitinib acesulfamate, and combinations thereof, with one or more pharmaceutically acceptable excipients. Preferably, the pharmaceutical composition comprises one or more crystalline form(s) of a ruxolitinib salt selected from the group consisting of ruxolitinib mesylate Form APO-I, ruxolitinib edisylate, Form APO-I, ruxolitinib napadisylate Form APO-I, ruxolitinib acesulfamate Form APO-I, ruxolitinib acesulfamate Form APO-II, and combinations thereof. In an embodiment, the pharmaceutical composition is a solid dosage form suitable for oral administration, such as a capsule, tablet, pill, powder or granulate. Preferably, the solid dosage form is a tablet. In another embodiment, the pharmaceutical composition is a cream suitable for topical administration, such as an emulsion. Preferably, the cream is an oil-in-water emulsion. Preferably, the pharmaceutical composition provides a dose of one or more ruxolitinib salt(s) selected from the group consisting of ruxolitinib mesylate, ruxolitinib edisylate, ruxolitinib napadisylate, or ruxolitinib acesulfamate, such that the total is equivalent to the 5 mg, 10 mg, 15 mg, 20 mg, or 25 mg of ruxolitinib free base found in JAKAFI® drug products or the 15 mg of ruxolitinib free base found in every gram of OPZELURA® drug products.

Suitable pharmaceutically acceptable excipients for use in solid dosage forms are preferably inert with respect to the ruxolitinib salts of the present invention, and may include, for example, one or more excipients selected from binders such as lactose, starches, modified starches, sugars, gum acacia, gum tragacanth, guar gum, pectin, wax binders, microcrystalline cellulose, methylcellulose, carboxymethylcellulose, hydroxypropyl methylcellulose, hydroxyethyl cellulose, hydroxypropyl cellulose, copolyvidone, gelatine, polyvinylpyrrolidone (PVP) and sodium alginate; fillers or diluents such as lactose, sugar, starches, modified starches, mannitol, sorbitol, inorganic salts, cellulose derivatives (e.g., microcrystalline cellulose, cellulose), calcium sulphate, xylitol and lactitol; disintegrants such as croscarmellose sodium, crospovidone, polyvinylpyrrolidone, sodium starch glycollate, corn starch, microcrystalline cellulose, hydroxypropyl methylcellulose and hydroxypropyl cellulose; lubricants such as magnesium stearate, magnesium lauryl stearate, sodium stearyl fumarate, stearic acid, calcium stearate, zinc stearate, potassium benzoate, sodium benzoate, myristic acid, palmitic acid, mineral oil, hydrogenated castor oil, medium-chain triglycerides, poloxamer, polyethylene glycol and talc; and dispersants or solubility enhancing agents, such cyclodextrins, glyceryl monostearate, hypromellose, meglumine, Poloxamer, polyoxyethylene castor oil derivatives, polyoxyethylene stearates, polyoxylglycerides, povidone, and stearic acid. Other excipients including preservatives, stabilisers, anti-oxidants, silica flow conditioners, antiadherents or glidants may be added as required. Other suitable excipients and the preparation of solid oral dosage forms are well known to person of skill in the art, and is described generally, for example, in Remington The Science and Practice of Pharmacy 21^{st} Edition (Lippincott Williams & Wilkins: Philadelphia; 2006; Chapter 45).

Optionally, when the pharmaceutical compositions are solid dosage forms, the solid dosage forms may be prepared with coatings, such as enteric coatings and extended-release coatings, using standard pharmaceutical coatings. Such coatings, and their application, are well known to persons skilled in the art, and are described, for example, in Remington The Science and Practice of Pharmacy 21^{st} Edition (Lippincott Williams & Wilkins: Philadelphia; 2006; Chapter 46).

Alternatively, the ruxolitinib salts and crystalline forms of the present invention may be formulated as a topical cream, such as an oil-in-water emulsion as described in, for example, WO 2011/146808 A1.

### EXAMPLES

The following non-limiting examples are illustrative of some of the aspects and embodiments of the invention described herein.

The ruxolitinib free base used as a starting material in the following examples was amorphous. Acesulfame used as starting material in the following examples was obtained by stirring an ethyl acetate suspension of acesulfame potassium and a stoichiometric amount of 85% phosphoric acid for about 3 hours, filtering the organic layer and concentrating the solution to dryness.

### PXRD Analysis:

PXRD diffractograms were recorded on a Bruker D8 Discover powder X-ray diffractometer (Bruker AXS LLC, Karlsruhe, Germany). The generator was a Incoatec Microfocus Source (IμS) Cu tube (λ=1.54060 Å) with a voltage of 50 kV and current of 1.00 mA, using a divergence slit of 0.1 mm and collimator of 2.0 mm. For each sample, two frames were collected using a still scan with a PILATUS3 R 100K-A detector at the distance of 294.2 mm from the sample. Raw data were evaluated using the program DIFFRAC.EVA (Bruker AXS LLC, Karlsruhe, Germany).

### Differential Scanning Calorimetry Analysis:

The DSC thermogram was collected on a Mettler-Toledo 821e instrument. Each sample (1-2.5 mg) was weighed into a 40 μL aluminum pan and was crimped closed with an aluminum lid having a 50 μm pinhole. The sample was analyzed under a flow of nitrogen (60±2 mL/min) at a scan rate of 10° C./minute between 25° C. and 280° C.

### Analysis Method for Determining the Chromatographic Purity of Ruxolitinib Mesylate

The method shown in Table 6 was used to determine the chromatographic purity of a sample of ruxolitinib mesylate as provided in Example 1.1.

TABLE 6
HPLC method for the determination of chromatographic
purity of Ruxolitinib Mesylate.

| Instrument | HPLC with variable wavelength UV-Vis detector | | |
|---|---|---|---|
| | or diode array detector (DAD), auto injector, | | |
| | sample cooler, column heater and suitable | | |
| | data processor/recorder | | |
| Column | Agilent Eclipse XDB-C18, 25 cm × 4.6 mm, 5 μm | | |
| Column Temp. | 25° C. | | |
| Sample temp. | 5° C. | | |
| Mobile phases | Solution A: Dissolve 2.7 g of KH₂PO₄ into 2 L of water. | | |
| | Adjust the pH to 5.5 +/− 0.05 with diluted KOH. | | |
| | Solution B: Mix 500 mL of methanol with 500 mL | | |
| | acetonitrile. | | |
| | Mobile Phase A: Mix 950 mL Solution A with 50 mL Solution | | |
| | B. Filter and de-aerate. | | |
| | Mobile Phase B: Mix 450 mL Solution A with 550 mL | | |
| | Solution B. Filter and de-aerate. | | |
| | Time | % mobile phase A | % mobile phase B |
| Gradient | 0.0 | 98 | 2 |
| Program | 2 | 98 | 2 |
| | 8 | 40 | 60 |
| | 30 | 0 | 100 |
| | 35 | 0 | 100 |
| | 36 | 98 | 2 |
| | 45 | 98 | 2 |

| | |
|---|---|
| Flow rate | 1.0 mL/minute |
| Injection volume | 10 μL |
| Detector | 220 nm |
| Run time | 45 minutes |
| Ruxolitinib | 26.0 minutes |
| Retention Time | |
| Sample prep. | Weigh out accurately an amount of sample equivalent to |
| | about 40 mg of ruxolitinib and transfer into a 20 mL |
| | volumetric flask. Dissolve in sample solvent (500 mL |
| | solution B with 500 mL water and 1.0 mL concentrated HCl), |
| | sonicate if necessary and dilute to volume with sample |
| | solvent, then mix. Do not filter. |

### Example 1: Preparation of Ruxolitinib Mesylate Form APO-I

To a solution of ruxolitinib free base (300 mg) in acetone (5 mL) was added methanesulfonic acid (0.11 mL), and the resulting solution was stirred at room temperature for 16 hours, during which precipitation occurred. The solids were collected by vacuum filtration, washed with acetone (3×0.7 mL), and dried in vacuo at room temperature for approximately 24 hours. Ruxolitinib mesylate Form APO-I was obtained as a white solid (215 mg). ¹H NMR analysis of the solid (DMSO-d₆) revealed a molar ratio of ruxolitinib:methanesulfonic acid of approximately 1:1. The PXRD diffractogram and DSC thermogram of a sample prepared by this method are shown in FIG. 1 and FIG. 6, respectively.

¹H NMR (300 MHZ, DMSO-d₆): 13.26 (s, 1H), 9.14 (s, 1H), 9.01 (s, 1H), 8.65 (s, 1H), 8.00 (t, J=2.9 Hz, 1H), 7.36 (t, J=1.8 Hz, 1H), 4.65 (m, 1H), 3.20-3.37 (m, 2H), 2.45 (m, 1H), 2.37 (s, 3H), 1.83 (m, 1H), 1.12-1.71 (m, 7H).

### Example 1.1: Preparation of Ruxolitinib Mesylate Form APO-I

To a solution of ruxolitinib free base (13.5 g) in a mixture of ethyl acetate/acetone (180 mL/72 mL, containing 4.7 wt % water) was added water (1.32 g), followed by methanesulfonic acid (4.13 g, 0.97 mol equivalents), in two approximately equal portions. After the first portion of acid, added over a period of 3 minutes, small oil droplets were formed, producing a cloudy suspension. After approximately 5 minutes, the oil hardened into solid particles, with no material adhered to the walls of the flask. The remaining acid was added dropwise over 8 minutes, after which agitation was continued for approximately 5 hours. The solids were then collected by vacuum filtration, washed with acetone (2×30 g) and dried in vacuo at room temperature overnight to afford ruxolitinib mesylate Form APO-I as a white solid (15.9 g, 89.5% yield). PXRD of the sample was consistent with FIG. 1. ¹H NMR analysis of the solid (DMSO-d₆) showed solvent levels of about 481 ppm of acetone and 469 ppm of ethyl acetate. HPLC analysis of the sample according to the method described above showed a purity of >99.9 a % with total impurities of 0.05 a %. A sample prepared by this method had a water content (Karl Fischer method) of 0.04 wt %, consistent with an anhydrate. The sample also displayed high solubility across a range of pH values (1.2, 4.5 and 6.8).

### Example 2: Preparation of Ruxolitinib Edisylate Form APO-I

To a solution of ruxolitinib free base (300 mg) in acetone (5 mL) was added 1,2-ethanedisulfonic acid dihydrate (120 mg) and water (0.5 mL), and the resulting solution was stirred at room temperature for 16 hours, during which precipitation occurred. The solids were collected by vacuum filtration, washed with acetone (3×0.7 mL), and dried in vacuo at room temperature for approximately 24 hours. Ruxolitinib edisylate Form APO-I was obtained as a white solid (237 mg). ¹H NMR analysis of the solid (DMSO-d₆) revealed a molar ratio of ruxolitinib:1,2-ethanedisulfonic acid of approximately 1:1. The PXRD diffractogram and DSC thermogram of a sample prepared by this method are shown in FIG. 2 and FIG. 7, respectively. TGA (25-360° C. @10° C./min; 85 mL/min N₂ flow) of the sample showed insignificant weight loss consistent with an anhydrous material.

¹H NMR (300 MHZ, DMSO-d₆): 13.27 (s, 1H), 9.15 (s, 1H), 9.01 (s, 1H), 8.65 (s, 1H), 8.01 (t, J=2.9 Hz, 1H), 7.37 (t, J=1.8 Hz, 1H), 4.65 (m, 1H), 3.19-3.37 (m, 2H), 2.72 (s, 2H), 2.42 (m, 1H), 1.84 (m, 1H), 1.13-1.71 (m, 7H).

### Example 3: Preparation of Ruxolitinib Napadisylate Form APO-I

To a solution of ruxolitinib free base (306 mg) in acetone (3.5 mL) was added 1,5-naphthalenedisulfonic acid tetrahydrate (190 mg) and water (0.5 mL), and the resulting solution was stirred at 40° C. for 1 h, during which precipitation occurred. The resulting suspension was stirred at room temperature for 4 hours, after which the solids were collected by vacuum filtration, washed with acetone (2×0.7 mL), and dried in vacuo at room temperature for approximately 16 hours. Ruxolitinib napadisylate Form APO-I was obtained as a white solid (425 mg). ¹H NMR analysis of the solid (DMSO-d₆) revealed a molar ratio of ruxolitinib:1,5-naphthalenedisulfonic acid of approximately 1:1. The PXRD diffractogram and DSC thermogram of a sample prepared by this method are shown in FIG. 3 and FIG. 8, respectively. TGA (25-360° C. @10° C./min; 85 mL/min N₂ flow) of the sample showed a weight loss of approximately 3.0% between 50° C. and 130° C., which may be consistent with a monohydrate.

¹H NMR (300 MHZ, DMSO-d₆): 13.22 (s, 1H), 9.11 (s, 1H), 9.00 (s, 1H), 8.87 (d, J=8.4 Hz, 1H), 8.63 (s, 1H), 7.98 (t, J=2.9 Hz, 1H), 7.94 (d, J=7.1 Hz, 1H), 7.41 (dd, J=8.4, 7.2 Hz, 1H), 7.35 (m, 1H), 4.64 (m, 1H), 3.19-3.36 (m, 2H), 2.44 (m, 1H), 1.83 (m, 1H), 1.12-1.70 (m, 7H).

### Example 4: Preparation of Ruxolitinib Acesulfamate Form APO-I

To a solution of ruxolitinib free base (800 mg) in ethyl acetate (18 mL) was added acesulfame (450 mg), and the resulting solution was stirred at 65° C. for 1 hour, during which precipitation occurred. The resulting suspension was stirred at room temperature for approximately 16 hours, after which the solids were collected by vacuum filtration, washed with ethyl acetate (2×5 mL), and dried in vacuo at room temperature for approximately 16 hours. Ruxolitinib acesulfamate Form APO-I was obtained as a white solid (1.14 g). ¹H NMR analysis of the solid (DMSO-d₆) revealed a molar ratio of ruxolitinib:acesulfame of approximately 1:1. The PXRD diffractogram and DSC thermogram of a sample prepared by this method are shown in FIG. 4 and FIG. 9, respectively.

¹H NMR (300 MHZ, DMSO-d₆): 12.77 (s, 1H), 8.98 (s, 1H), 8.87 (s, 1H), 8.52 (s, 1H), 7.83 (d, J=2.5 Hz, 1H), 7.20 (d, J=2.0 Hz, 1H), 5.65 (d, J=0.9 Hz, 1H), 4.60 (m, 1H), 3.17-3.35 (m, 2H), 2.44 (m, 1H), 2.04 (s, 3H), 1.83 (m, 1H), 1.13-1.69 (m, 7H).

### Example 5: Preparation of Ruxolitinib Acesulfamate Form APO-II

Ruxolitinib free base (504 mg) and acesulfame (351 mg) were dissolved in a mixture of isopropanol (5 mL) and water (2 mL), and the resulting solution was stirred at room temperature for 5 hours, during which precipitation occurred. The solids were collected by vacuum filtration, washed with isopropanol (1×0.7 mL), and dried in vacuo at room temperature for approximately 16 hours. Ruxolitinib acesulfamate Form APO-II was obtained as a white solid (459 mg). ¹H NMR analysis of the solid (DMSO-d₆) revealed a molar ratio of ruxolitinib:acesulfame of approximately 1:1. The PXRD diffractogram and DSC thermogram of a sample prepared by this method are shown in FIG. 5 and FIG. 10, respectively.

¹H NMR (300 MHZ, DMSO-d₆): 12.77 (s, 1H), 8.98 (s, 1H), 8.87 (s, 1H), 8.52 (s, 1H), 7.83 (d, J=2.5 Hz, 1H), 7.20 (d, J=2.0 Hz, 1H), 5.65 (d, J=0.9 Hz, 1H), 4.60 (m, 1H), 3.17-3.35 (m, 2H), 2.44 (m, 1H), 2.04 (s, 3H), 1.83 (m, 1H), 1.13-1.69 (m, 7H).

### Example 6: Comparative Intrinsic Dissolution Testing

Intrinsic dissolution rate (IDR) measurements were performed using a Wood apparatus. Samples were prepared by compressing 250-300 mg sample at 1.5 metric tons for 1 minute. A dissolution medium consisting of 900 mL deionized water, and rotation speed of 75 rpm, was used for each experiment. Results are provided in Table 7.

TABLE 7
Comparative intrinsic dissolution rates for salts of the invention
with the ruxolitinib phosphate described in WO 2008/157208 A2

| | Intrinsic Dissolution Rate |
|---|---|
| Form | (mg min⁻¹ cm⁻²) |
| Ruxolitinib phosphate (Prior Art) | 0.0545 |
| Ruxolitinib edisylate Form APO-I | 0.1711 |
| Ruxolitinib acesulfamate Form | 0.2261 |
| APO-I | |
| Ruxolitinib acesulfamate Form | 0.0767 |
| APO-II | |

### Example 7: Preparation of Ruxolitinib Acesulfamate Form APO-II

Ruxolitinib free base (504 mg) and acesulfame (351 mg) were dissolved in a mixture of isopropanol (5 mL) and water (2 mL), and the resulting solution was stirred at room temperature for 5 hours, during which precipitation occurred. The solids were collected by vacuum filtration, washed with isopropanol (1×0.7 mL), and dried in vacuo at room temperature for approximately 16 hours. Ruxolitinib acesulfamate Form APO-II was obtained as a white solid (459 mg). ¹H NMR analysis of the solid (DMSO-d₆) revealed a molar ratio of ruxolitinib:acesulfame of approximately 1:1. The PXRD diffractogram and DSC thermogram of a sample prepared by this method are shown in FIG. 5 and FIG. 10, respectively.

1. A mesylate salt of ruxolitinib.

2. The mesylate salt of ruxolitinib of claim 1, wherein the molar ratio of ruxolitinib to methanesulfonic acid is approximately 1:1.

3. The mesylate salt of claim 2, characterized by a PXRD diffractogram comprising peaks, expressed in degrees 2θ (±0.2°), at 6.8°, 8.0°, and 11.1°.

4. The mesylate salt of claim 3, further comprising at least three peaks in the PXRD diffractogram, expressed in degrees 2θ (±0.2°), selected from the group consisting of: 11.7°, 13.1°, 16.2°, 17.8°, 19.3°, and 22.5°.

5. The mesylate salt of claim 3, further comprising peaks in the PXRD diffractogram, expressed in degrees 2θ (±0.2°), at 11.7°, 13.1°, 16.2°, 17.8°, 19.3°, and 22.5°.

6. The mesylate salt of claim 1, providing a PXRD diffractogram comprising peaks in substantially the same positions (±0.2° 2θ) as those shown in FIG. 1.

7. The mesylate salt of claim 3, characterized by a DSC thermogram comprising an endothermic peak with a peak onset at approximately 163° C. and a peak maximum at approximately 166° C.

8. The mesylate salt of claim 3, characterized by a DSC thermogram that is substantially the same in appearance as the DSC thermogram provided in FIG. 6.

9. A salt of ruxolitinib selected from the group consisting of an edisylate salt of ruxolitinib, a napadisylate salt of ruxolitinib and an acesulfame salt of ruxolitinib.

10. The edisylate salt of ruxolitinib of claim 9, wherein the molar ratio of ruxolitinib to 1,2-ethanedisulfonic acid is approximately 1:1; and the edisylate salt is characterized by a PXRD diffractogram comprising peaks, expressed in degrees 2θ (±0.2°), at 4.1°, 8.3°, and 15.7°.

11. (canceled)

12. The edisylate salt of claim 10,
a) further comprising at least three peaks in the PXRD diffractogram, expressed in degrees 2θ (±0.2°), selected from the group consisting of: 7.9°, 9.5°, 16.2°, 18.4°, 20.6, 17.7°, and 22.3°; or
b) further comprising peaks in the PXRD diffractogram, expressed in degrees 2θ (±0.2°), at 7.9°, 9.5°, 16.2°, 18.4°, 20.6, 17.7°, and 22.3°; or
c) providing a PXRD diffractogram comprising peaks in substantially the same positions (±0.2° 2θ) as those shown in FIG. 2; or
d) characterized by a DSC thermogram comprising an endothermic peak with a peak onset at approximately 148° C. and a peak maximum at approximately 157° C.; or
e) characterized by a DSC thermogram that is substantially the same in appearance as the DSC thermogram provided in FIG. 7.

13-17. (canceled)

18. The napadisylate salt of ruxolitinib of claim 472, wherein the molar ratio of ruxolitinib to 1,5-naphthalenedisulfonic acid is approximately 1:1 and the napadisylate salt is characterized by a PXRD diffractogram comprising peaks, expressed in degrees 2θ (±0.2°), at 7.2°, 9.5°, and 15.7°.

19. (canceled)

20. The napadisylate salt of claim 18,
a) further comprising at least three peaks in the PXRD diffractogram, expressed in degrees 2θ (±0.2°), selected from the group consisting of: 12.0°, 14.3°, 16.7°, 17.3°, 18.8°, and 19.4°; or
b) further comprising peaks in the PXRD diffractogram, expressed in degrees 2θ (±0.2°), at 12.0°, 14.3°, 16.7°, 17.3°, 18.8°, and 19.4°; or
c) providing a PXRD diffractogram comprising peaks in substantially the same positions (±0.2° 2θ) as those shown in FIG. 3; or
d) characterized by a DSC thermogram comprising an endothermic peak with a peak onset at approximately 154° C. and a peak maximum at approximately 165° C.; or
e) characterized by a DSC thermogram that is substantially the same in appearance as the DSC thermogram provided in FIG. 8; or
f) having a weight percentage of water of between approximately 2.4 wt. % and 3.6 wt. %; or
g) having a weight percentage of water of between approximately 2.8 wt. % and 3.2 wt. %.

21-27. (canceled)

28. The acesulfamate salt of ruxolitinib of claim 9, wherein the molar ratio of ruxolitinib to acesulfame is approximately 1:1.

29. The acesulfamate salt of claim 28, characterized by a PXRD diffractogram comprising peaks, expressed in degrees 2θ (±0.2°), at 4.1°, 9.0°, and 22.5°.

30. The acesulfamate salt of claim 29,
a) further comprising at least three peaks in the PXRD diffractogram, expressed in degrees 2θ (±0.2°), selected from the group consisting of: 8.4°, 12.8°, 13.5°, 15.8°, 20.3°, and 25.5°; or
b) further comprising peaks in the PXRD diffractogram, expressed in degrees 2θ (±0.2°), at 8.4°, 12.8°, 13.5°, 15.8°, 20.3°, 20.3°, and 25.5°; or
c) providing a PXRD diffractogram comprising peaks in substantially the same positions (±0.2° 2θ) as those shown in FIG. 4; or
d) characterized by a DSC thermogram comprising an endothermic peak with a peak onset at approximately 112° C. and a peak maximum at approximately 121° C.; or
e) characterized by a DSC thermogram that is substantially the same in appearance as the DSC thermogram provided in FIG. 9.

31-34. (canceled)

35. The acesulfamate salt of claim 28, characterized by a PXRD diffractogram comprising peaks, expressed in degrees 2θ (±0.2°), at 10.8°, 11.4°, and 13.0°.

36. The acesulfamate salt of claim 35,
a) further comprising at least three peaks in the PXRD diffractogram, expressed in degrees 2θ (±0.2°), selected from the group consisting of: 4.4°, 8.7°, 14.0°, 15.9°, 20.5°, and 21.6°; or
b) further comprising peaks in the PXRD diffractogram, expressed in degrees 2θ (±0.2°), at 4.4°, 8.7°, 14.0°, 15.9°, 20.5°, and 21.6°; or
c) providing a PXRD diffractogram comprising peaks in substantially the same positions (±0.2° 2θ) as those shown in FIG. 5; or
d) characterized by a DSC thermogram comprising an endothermic peak with a peak onset at approximately 77° C. and a peak maximum at approximately 81° C.; or
e) characterized by a DSC thermogram that is substantially the same in appearance as the DSC thermogram provided in FIG. 10.

37-40. (canceled)

41. A pharmaceutical composition comprising the salt of ruxolitinib according to claim 1, and one or more pharmaceutically acceptable excipients.

42. The pharmaceutical composition of claim 41, wherein the pharmaceutical composition is a tablet.

43. The pharmaceutical composition of claim 41, wherein the pharmaceutical composition is an oil-in-water cream emulsion.

44. A method of treating a disorder selected form the group consisting of acute graft versus host disease, polycythemia vera, and myelofibrosis comprising administering the pharmaceutical composition of claim 41 to a human subject in therapeutically effective amount for the treatment of a disorder selected from the group consisting of acute graft versus host disease, polycythemia vera, and myelofibrosis.

45. A method of treating atopic dermatitis comprising administering the pharmaceutical composition of claim 43 to skin of a human subject in a therapeutically effective amount for the treatment of atopic dermatitis.
